(19) United States
(12) Patent Application Publication
(10) Pub. No.: US 2024/0016385 A1
(43) Pub. Date: Jan. 18, 2024

(54) NONINVASIVE MEASUREMENT OF BIOMARKER CONCENTRATION

(71) Applicant: SIATLAB GMBH, Graz (AT)

(72) Inventors: Daniel TEMENT, Markovci (SI); Tadej TOFANT, Ptuj (SI)

(21) Appl. No.: 18/245,521

(22) PCT Filed: Sep. 15, 2021
(86) PCT No.: PCT/EP2021/075352

§ 371 Date: Mar. 15, 2023

(30) Foreign Application Priority Data
Sep. 16, 2020 (DE) 10 2020 124 166.6

(51) Int. Cl. A61B 5/00; A61B 5/145; A61B 5/1455

(52) U.S. Cl. CPC A61B 5/0053; A61B 5/14532; A61B 5/14552; A61B 5/6898

(57) ABSTRACT

The present disclosure relates to a device for determining a biomarker concentration in a blood of a body part under consideration of the physiological constitution of the body part. The device comprises a light source for radiating first light waves to the body part, a detector unit for measuring the reflected first light waves reflected from the body part and a processing unit coupled to the detector unit for receiving the measured first light waves. The processing unit is configured to determine, at an occurrence of a first specific signal section in a signal profile of the reflected first light waves during a predefined pressure variation applied to the body part by the detector unit, at least one characteristic value comprising the signal strength of the reflected first light waves, wherein the at least one characteristic value at the specific first signal section of the reflected first light waves is representative of a physiological constitution of the body part, such that a biomarker concentration in the blood is determinable.

This application is a national phase of international application WO 2022/058363 A1 claiming benefit of the filing date of the German Patent Application No. 10 2020 124 166.6 filed Sep. 16, 2020, the disclosure of which is hereby incorporated herein by reference.

### TECHNICAL FIELD

The present invention relates to a device for determining a biomarker concentration in the blood of a body part, such as a finger, under consideration of the physiological constitution of the body part. Furthermore, the present invention relates to a method for determining a biomarker concentration in the blood of a body part, such as a finger, under consideration of the physiological constitution of the body part.

### ART BACKGROUND

In order to measure biomarker concentrations, for example a concentration of a blood glucose level, invasive measuring methods and respective measurement devices exist. Blood is taken from the tissue of a person and the respective blood is analyzed in order to determined concentration of the respective biomarker concentration.

Furthermore, noninvasive measurement methods are known. For example, devices exist which radiates respective light, such as infrared light, with defined wavelengths into tissue of the person. On the basis of the measured reflected light, it is generally possible to determine the occurrence and the concentration of the specific bio marker. However, conventional noninvasive measurement methods are not very precise due to the large variety of the physiological constitution the body part and many other environmental measurement parameters.

One reason for the inaccurate measurement results is that the physiology of a measured body part, such as a finger, varies and changes very quickly over time. The physiology of the measured part may be defined for example by the temperature of the finger, the skin thickness, the blood circulation of the subcutaneous tissue, the subcutaneous thickness, the depth of bone, the skin color and e.g. the skin moisture.

Furthermore, in conventional measurement methods, the undefined pressure of the body part, such as a finger, to a respective detection device may lead to an inaccurate measurement of the respective bio marker concentration.

For example, WO 2016/068589 A1 discloses a glucose measurement apparatus for measuring a blood glucose level based on infrared spectroscopy. In order to determine a measurement error caused by unknown pressure between the body part and a detection unit, a pressure sensor is used to measure the pressure applied from the body part to the apparatus.

WO 00/21437 A2 discloses an infrared glucose measurement system using an attenuated total internal reflectance spectroscopy. The measurement system comprises a pressure maintaining member for maintaining a predefined pressure between the body part and a respective detection plate of the measurement system.

Hence, either noninvasive measurement devices are inaccurate, so that reliable measurement results are not possible, or complex devices have to be provided for pre-determining a specific pressure.

### SUMMARY

There may be a need to provide a simple measurement device which provides additionally a high accuracy of a noninvasive measurement of the biomarker in the blood of the person.

According to first aspect of the present disclosure a device for determining a biomarker concentration in a blood of a body part, for example a finger of a person, under consideration of the physiological constitution of the body part is presented. The device comprises a light source for radiating first light waves to the body part, a detector unit for measuring the reflected first light waves reflected from the body part. Furthermore, the device comprises a processing unit coupled to the detector unit for receiving the measured first light waves.

The processing unit is configured to determine, at an occurrence of a first specific signal section in a signal profile of the reflected first light waves during a predefined pressure variation applied to the body part by the detector unit, at least one characteristic value comprising the signal strength of the reflected first light waves. The at least one characteristic value at the specific first signal section of the reflected first light waves is representative of a physiological constitution of the body part, such that a biomarker concentration in the blood is determinable.

According to a further aspect of the present disclosure, a method of determining a biomarker concentration in a blood of a body part under consideration of the physiological constitution of the body part is presented. The method comprises the step of radiating first light waves to the body part, measuring the reflected first light waves reflected from the body part, and determining, at an occurrence of a first specific signal section in a signal profile of the reflected first light waves during a predefined pressure variation applied to the body part by the detector unit, at least one characteristic value comprising the signal strength of the reflected first light waves, wherein the at least one characteristic value at the specific first signal section of the reflected first light waves is representative of a physiological constitution of the body part, such that a biomarker concentration in the blood is determinable.

The device may be portable handheld device, in particular a smartphone, a tablet computer or a notebook.

The determined biomarker may be Glucose, C-Reactive Protein (CRP), Hemoglobin (HBC), Cholesterol, LDL, HDL, Fibrinogen and/or Bilirubin.

The light source is configured to radiate light with the first wavelength or with a predefined plurality of further wavelengths to the body part. The light source may comprise one or a plurality of LEDs. Specifically, the first wavelength may have for example 420 nm to 490 nm (blue light), 490 nm to 575 nm, in particular 530 nm (green light), 585 nm to 750 nm, in particular 660 nm (red light) and 780 nm und 1000 nm, in particular 960 nm (infrared IR light).

The detector unit may comprise a photodiode which is configured to measure all opposed described spectra used for the respective radiated wavelengths. Specifically, the detector unit may detect a picture or the multiple spectra between 410 nm and 1090 nm, for example.

The detector unit may measure the of illuminance in [Lux] of the received reflected wavelength. Next, in a signal acquisition process the measured illuminance is transferred to a Row-ADC-signal having e.g. the unit [nA] (nano Amperes). A value for the signal strength in nA may be for example between 0 and 224 000 nA. However, the values depend on the used sensor (detector unit) and thus may vary when using different sensors.

The processing unit may comprise a processor for controlling the light source and the detector unit. Specifically, the processing unit may comprise for example an oscillator, a led driver, a temperature sensor and a data register. Furthermore, the processing to transfer data via standard buses such as I2C or SPI communications or similar.

Furthermore, the device may comprise a display unit for displaying the measurement results and/or for giving instruction to the user. Additionally, the display unit may form an input unit, such as a touchscreen.

The quality and the quantity of the signal strength of the reflected and hence detected wavelengths is dependent on the physiological constitution of the body part and specifically of the pressure, by which the detection unit is pressed onto the body part. By the approach of the present disclosure it has found out, that independent of the knowledge of measured pressure values applied onto the body part and the physiological constitution of the body part, the detected signals during a pre-determined pressure variation can be representative for a quantity of the biomarker concentration.

The pressure variation may be for example an increase or decrease of the pressure in a certain time interval. The pressure variation may be independent from an initial pressure and an end pressure of the pressure variation. For example, a (one) predetermined pressure variation may be an increase or decrease of the pressure within a timespan of e.g. 10 to 20 seconds.

During the predefined pressure variation, it has found out, that in the signal profile of the detected reflected light waves during the predefined pressure radiation a specific signal section (e.g. a certain shape) exists. Furthermore, it has found out, that the specific signal section and its respective characteristic value (e.g. the strength of the detected signal at the specific signal section) is indicative of a certain biomarker (e.g. glucose) and its respective concentration. Furthermore, it has found out, that the characteristic values derived from signals of the specific signal sections may define a specific physiological constitution of the body part at the time of measurement. For example, if the body part is a finger and the finger is pressed onto the detection unit during a predefined pressure variation, a local maximum as signal section of a detected signal profile may be indicative of the amount of tissue between the surface of the finger and the bone of the finger. Hence, the thickness of the tissue between the bone and the surface of the finger can be derived which also influence the measurement result of the concentration of the biomarker.

Specific points and specific signal sections, respectively, in the signal profile may be a plateau of the signal function, a function break (a sudden change in the slope of the function), a maximum and minimum of the signal function.

Hence, because a pressure variation without predefining an initial pressure can be conducted by a user without measuring a total amount of pressure at the certain time point, by the present disclosure complex pressure sensors are not necessary. Furthermore, the determining of the characteristic value of specific signal sections during the predefined pressure variation leads to a more accurate determination of a biomarker concentration, a more accurate measurement system is provided.

The determined characteristic value at a specific signal section of the reflected light waves may be compared with existing models comprising the information of a respective biomarker concentration in the blood on the certain characteristic value of a specific signal section. The existing models are defined for example in clinical studies and laboratory studies. For example, if the biomarker is glucose, the glucose level and the physiological constitution of the plurality of persons can be measured for example invasively. For example, the exact glucose level may be measured for a specific physiological constitution of the user by an oral glucose tolerance test (OGTT). For measurement value of the glucose level, a specific characteristic value of a specific signal section in the signal profile of the reflected light waves can be determined. Hence, a database comprising a plurality of nominal values can be provided to which the measured characteristic values of the inventive device can be compared with in order to determined specific biomarker concentration the blood. In fact, a plurality of the specific signal sections under a plurality of different light waves can be derived for a specific biomarker concentration under consideration of a specific physiological constitution. For example, as described below, statistic methods based on defined regressors and regressor relations, respectively, can be used in order to further increase the accuracy of the determined concentration level of the biomarker.

According to further exemplary embodiment, the characteristic value further comprises the value of the slope of the signal profile at the occurrence of a specific signal section during the predefined pressure variation applied to the body part by the detector unit.

According to further exemplary embodiment the specific signal section is defined by a characteristic slope, by a plateau of the signal function, a saltus of the signal function, an inflection point, a minimum, in particular local minimum of the signal function, and a maximum, in particular local maximum of the signal function. Hence, during the predefined pressure variation, the respective signal profiles of the reflected light waves comprise for example the above listed specific signal sections that are indicative for the biomarker concentration and the physiological constitution of the body part.

According to further exemplary embodiment, the processing unit is configured to determine on a basis of a plurality of repeated predefined pressure variations occurrences of the first specific signal section in a signal profile of the reflected first light waves for each conducted pressure variation. The processing unit is further configured to determine respective characteristic values of the first specific signal section in each predefined pressure variations and to determine a mean characteristic value of the first specific signal section determined in the predefined pressure variations. Hence, if a pressure variation is an increasing of the pressure for 10 seconds and if the user increases the pressure only for 5 seconds, error measurements may occur. However, by providing a plurality of measurements during a plurality of pressure variations, the mean value of all measurements reduces the impact of one error measurement.

According to further exemplary embodiment, the at least one determined characteristic value defines at least one respective characteristical regressor (Rc). The processing unit is configured to determine a regressor relation (RR) on the basis of the at least one determined characteristical regressor (Rc), wherein the regressor relation is correlatable to a biomarker concentration in the blood, such that a determined value of the regressor relation is indicative to a value of the biomarker concentration.

The characteristic values at the appearance of this specific signal sections defines the first list of regressors—characteristical regressors (Rc). This list of regressors Rc may be used for generating a regressor relation, which can be correlated to a biomarker concentration. The regressor relation defines a mathematical relation between at least one characteristical regressor or the relation of the plurality of different characteristical regressors. By using statistical methods and machine learning, i.e. artificial intelligence (AI), specific regressor relations can be found out which are suitable for determining by its characteristic value a biomarker concentration for a specific physiological constitution on the basis of the specific signal sections of the signal profiles of reflected light waves under the predefined pressure variation.

The term “machine learning” may particularly denote the implementation of algorithms and/or statistical models that a processor (such as a computer system) may use to find out a respective regressor relation which matches at best the bio marker concentration under certain physiological constitution of the body part. By machine learning the best matching regressor relation may be found without using explicit instructions, relying on the patterns and inference instead. Machine learning may be considered as a subset of artificial intelligence. In particular, machine learning algorithms may build a mathematical model based regressor relation with respect to sample data (such as biomarker concentration measured under laboratory conditions, i.e. invasive or by an above described OGTT Test in case of Glucose as biomarker) in order to make predictions or decisions without being explicitly programmed to perform the task. Machine learning algorithms may be particularly appropriately applied in the evaluation of the regressors and the regressor relation being indicative of the specific signal sections in a signal profile of reflected wavelengths.

In an embodiment, the machine learning using at least one of the group consisting of Random Forest, Random Fern, Support Vector Machine, and a neural network, in particular a Convolutional Neural Network.

The term “Random Forest” may particularly denote an ensemble learning method for classification, regression and other tasks that operates by constructing a multitude of decision trees at training time and outputting the class that is the mode of the classes (which may be denoted as classification) or mean prediction (which may be denoted as regression) of the individual trees.

The term “Random Fern” may particularly denote a machine learning algorithm for matching the same elements between two images of the same scene, allowing to recognize an object (such as a solid pharmaceutical composition or part thereof) or trace it. Random Fern may be implemented as a classification method.

The term “Support Vector Machine” may particularly denote a supervised learning model with associated learning algorithms that analyze data used for classification and regression analysis. Given a set of training examples, each marked as belonging to one or the other of two categories, a Support Vector Machine training algorithm may build a model that assigns new examples to one category or the other. A Support Vector Machine model may be a representation of the examples as points in space, mapped so that the examples of the separate categories are divided by a clear gap that is as wide as possible. New examples may then be mapped into that same space and predicted to belong to a category based on the side of the gap on which they fall.

The term “neural network” (or artificial neural network) may particularly denote a computing system (which may be inspired by biological neural networks that constitute human or animal brains) which may learn to perform tasks by considering examples, generally without being programmed with task-specific rules. A neural network may identify a pattern without any prior knowledge of an object to be identified (for instance a coating of a solid composition). Additionally or alternatively, a neural network may automatically generate identifying characteristics from examples of training data that a neural network processes. A neural network may be based on a collection of connected units or nodes which may be denoted as artificial neurons. Each connection between different nodes can transmit a signal to other neurons. An artificial neuron that receives a signal may then process it and can signal neurons connected to it.

Hence, machine learning may be implemented in the evaluation of finding appropriate regressor relation. Detection data of the reflected wavelengths captured in laboratory conditions by a detection unit may be at least partially analyzed using machine learning. This may render it possible to obtain highly reliable information concerning regressor relations being indicative of a biomarker concentration under certain physiological constitutions of a specific body part (such as a finger).

It has turned out by the present disclosure that regressor relations indicative of the specific signal sections in a signal profile of reflected wavelengths are appropriate to be evaluated by machine learning, since such compositions (e.g. regressor relations of regressors of several signal sections in signal profiles of different wavelengths) may show a reliable prediction of a biomarker concentration.

The determined appropriate regressor relations for determining a biomarker concentration of a specific biomarker under certain physiological constitution of a specific body part (e.g. a finger, lips etc. of a person) may be correlated to laboratory measurement results of biomarker concentrations of a person in laboratory tests and stored in a respective data basis. Hence, upon measuring a certain characteristic value for the specific regressor relation, a respective concentration of biomarker can be determined by comparing to respective nominal values of the regressor relation in the databases without determining the physiological constitution of the user, since the influence of the actual biomarker concentration is already considered by the regressor relation.

According to further exemplary embodiment, the device further comprises a data unit comprising a data set of predefined regressor relations correlated to respective biomarker concentration. The control unit is further configured to compare the determined regressor relation to predefined regressor relations, wherein if the determined regressor relation is in the vicinity of the predefined regressor relation the biomarker concentration is derivable.

The data unit may be implemented in the device. However, the data unit may be realized by an input/output interface of the device and the data can be received and/or send to spaced apart data units which store the data. Hence, web-based application may be used, wherein the data are stored in (web) server or cloud server and the device receive and/or send the data via the internet or other network connections.

According to a further exemplary embodiment, the processing unit is further configured to determine, at an occurrence of a further first specific signal section in the signal profile of the reflected first light waves during the predefined pressure variation applied to the body part by the detector unit, at least one further characteristic value comprising a further signal strength of the first reflected first light waves, wherein the at least one further characteristic value at the further specific first signal section of the first reflected first light waves is representative of the physiological constitution of the body part, such that the biomarker concentration in the blood is determinable. The at least one determined further characteristic value of the further specific first signal section defines at least one respective further characteristical regressor (Rcf), wherein the regressor relation is further determined on the basis of the at least one determined further characteristical regressor (Rcf). By the exemplary embodiment it is outlined, that a signal profile of a reflected wavelength may have a plurality of specific signal sections which may be used as a regressor for defining a regressor relation. Hence, the regressor relation (RR) is formed by mathematical dependencies and relations of characteristical regressors (Rc) and further characteristical regressors (Rcf).

According to a further exemplary embodiment, the processing unit is further configured to determine at least one measurement value of the signal strength of the reflected first light waves during a, in particular constant, placement of the detector unit onto the body part (and hence almost constant pressure), wherein the measurement value defines at least one measurement regressor (Rm). The regressor relation (RR) is further determined on the basis of the at least one determined characteristical regressor (Rc, Rcf) and the at least one Measurement regressor (Rm).

It has turned out, that by additionally measuring the reflected wavelength during a placement of the detector onto the body part, i.e. under (almost) constant pressure, a characteristical value of the reflected signal of a specific wavelength taken under almost constant pressure may define a measurement regressor which can be used for normalization of the data with respect to the present physiological constitution of the body part and with respect to a calibration of the light source, e.g. the LEDs. The measurement regressor is additionally considered in the regressor relation so that an improved reference of the regressor relation to a nominal regressor relation indicative of a bio marker concentration can be achieved.

According to further exemplary embodiment, the light source is configured for radiating second light waves to the body part, wherein the detector unit is configured for measuring the reflected second light waves reflected from the body part. The detector unit is configured for receiving the measured second light waves. The processing unit is further configured to determine, at an occurrence of a second specific signal section in a second signal profile of the reflected second light waves during the predefined pressure variation applied to the body part by the detector unit, at least one further characteristic value comprising the signal strength of the reflected second light waves, wherein the at least one further characteristic value at the specific second signal section of the reflected second light waves is representative of the physiological constitution of the body part. The at least one determined further characteristic value defines at least one respective further characteristical regressor, wherein the regressor relation is further determined on the basis of the at least one determined further characteristical regressor (Rc2).

By the above described exemplary embodiment it is outlined, that a specific spectrum of different wavelengths can be radiated and received by the present device, such that the regressor relation is additionally formed by further characteristical regressors being indicative of signal sections of signal profiles of further different wavelengths.

Summarizing, during a measurement of reflected light of a body part under a predefined pressure variation on the body part, each reflected wavelength (for example red light, infrared light, blue light, green light etc.) has a specific signal profile under a specific pressure variation applied onto the body part. It has found out that each signal profile under pressure variation comprises a respective specific signal section in the signal profile of the reflected first light waves indicative of a physiological constitution and/or of a concentration of the measured biomarker.

At least one characteristic value comprising the signal strength at the occurrence of the first specific signal section during a predefined pressure variation applied to the body part by the photosensor can be derived. The characteristic values from this signal profile may comprise a signal strength at specific signal section and/or the value of the slope (derivation) at specific points.

According to the second finding of the present disclosure, it has found out, that a specific regressor relation of many regressors can be significantly better correlated to a biomarker concentration (e.g. glucose level) in the blood. The Specific regressor relation is obtained as mathematical relations of characteristical regressors (Rc) and e.g. measurement regressors (Rm) like: Rm1/Rc1, Rm2/Rc1, Rm1/Rc2, Rm1/In(Rc₁), In(Rm₁)/e^{Rc1} etc.

The regressors Rm (from second part of measurement under almost constant pressure) does not correlate good enough with biomarkers in the blood because of the lack of information regarding to specific physiology of the skin at the time of measurement.

By combining regressors Rm with regressors Rc the specific regressor relation RR can be created and they form e.g. input regressors (Ri). Input regressors Ri correlate significantly better with biomarkers concentrations in blood (e.g. glucose level). The procedure of mathematical correction of measurement regressors with characteristical regressors may be called Physiological Normalization.

Hence, the combination of the above-mentioned key findings results in a very accurate noninvasive measurement of a concentration of the bio marker, such as glucose, in the blood of the respective body part. Specifically, by the Physiological Normalization according to the measurement under predefined pressure variation, the physiological constitution of the body part at the time of measurement does not longer dramatically affect the quality of the measurement results at the time of measurement, since the measurements are normalized. Additionally, by using the specific regressor relations by measurement under almost constant pressure, a very exact correlation to the desired biomarker concentration is possible.

In fact, each wavelength (infrared, green, red etc.) defines under predefined pressure variation (first part of measurement) a specific signal profile and respective specific signal profile section. Hence, on basis of the many signal profiles and respective specific signal sections, a plurality of regressors can form a more complex specific regressor relation. Such complex specific regressor relations for a specific bio marker concentration can be formed by applying for example mathematical/statistical algorithms. Such complex specific regressor relations can be very successfully used as input parameters (regressors) for regression analysis with machine learning and artificial intelligence.

In an exemplary measuring procedure conducted by the inventive device, the first list of regressors Rc is received by pressing a photodetector during a predefined pressure interval (e.g. from minimum pressure to maximum pressure) to the body part (e.g. in finger) and the second list of regressors Rm is received by laying the photodetector onto the finger to provide an almost constant pressure.

Furthermore, the measurement cycles during a variety pressure and a constant pressure may be repeated several times in order to provide proper mean values for the regressors for improving the measurement quality. Furthermore, before conducting the measurement, a respective calibration of the light emitter and the respective light detector can be conducted.

The device may be a smartphone, or it can also function as a standalone device with properly added components such as processor, screen, power management, communication module, battery, charger, etc. The device may be in contact with the skin directly on the surface when the measurements is conducted. When measuring begins, the sensor first turns on the light source, e.g. the photodiode, and measures the current on the light source. In this way, the sensor may solve the problem of ambient light, a torque of electric current generated on the light source itself due to the effects of the environment or the physical parameters of the light source. Then, the device individually drives e.g. diodes of the light source with a frequency of e.g. 20 Hz to 100 Hz.

When the device is covered with the body part (e.g. the finger pad, preferably an index finger or a ring finger), a fixing element (such as a rubber ring, elastic or any other elastic, rope, fastener) of the device may be used to fix the body part to the device for more accurate measurement.

Next, the measurement of an individual person begins. Each person has a different skin type and other physiological properties which can be evaluated by the inventive device. According to the disclosure the skin surface is pushed with e.g. three consecutive pressures to the device, so that blood is squeezed out of the body part (e.g. the tip of the finger) and the body part slightly fades. Pressures occur e.g. in the sequences, first a gradual pressure increase to the point where the diode signals are no longer distinguishable, which lasts e.g. about 10 s (seconds), followed by a gradual release of the pressure of e.g. 5 s, and then the whole process can be repeated for example two times or more. Then, the body part may rest e.g. for 20 s onto the device under almost constant pressure.

Next, first the validity of the signal and its quality may be checked. Next, by the present disclosure the physiology of the skin of the finger may be considered based on the relationships between above described regressors and the regressor relation. The physiology of the body part is considered in the respective regressor relation. On the basis of the data of such measurement, it is possible to determine the actual skin and subcutaneous properties on the basis of the first part of the measurement under pressure variation and perform the physiological normalization (FN) for the second part of the, measurement under almost constant pressure. The physiological normalization is used to normalize the data of the second part of the measurement by translating the values to a neutral (universal) model (databases), where all the values obtained have e.g. the same scale (unit). Based on the data of the regressor relation, it is possible to determine the location of the actual measured regressor relation within the multidimensional space of the data bases of nominal regressors relations that are correlated to concentration of bio markers, e.g. blood sugar levels. The location of the measured regressor relation in the multidimensional space of the data bases is determined on the basis of clustering, which, based on the data of the e.g. the first part of the measurement, determines the location of the statistical model in the spectral space of the models. A more detailed classification of the measured regressor relation may be provided by checking the relationships between the signals of different wavelengths in a given measurement range.

According to still another exemplary embodiment of the disclosure, a program element (for instance a software routine, in source code or in executable code) is provided, which, when being executed by a processor, e.g. the processor unit (such as a microprocessor a CPU, a GPU, an FPGA or an ASCI), is adapted to control or carry out a method having the above mentioned features.

According to yet another exemplary embodiment of the disclosure, a computer-readable medium (for instance a CD, a DVD, a USB stick, a floppy disk, a hard disk, a flash drive or a Blu-ray disk) is provided, in which a computer program is stored which, when being executed by a processor (such as a microprocessor a CPU, a GPU, an FPGA or an ASCI), is adapted to control or carry out a method having the above mentioned features.

Data processing which may be performed according to embodiments of the disclosure can be realized by a computer program (e.g. by an application (app) installed in a smartphone), that is by software, or by using one or more special electronic optimization circuits, that is in hardware, or in hybrid form, that is by means of software components and hardware components.

It has to be noted that embodiments of the disclosure have been described with reference to different subject matters. In particular, some embodiments have been described with reference to apparatus type claims whereas other embodiments have been described with reference to method type claims. However, a person skilled in the art will gather from the above and the following description that, unless other notified, in addition to any combination of features belonging to one type of subject matter also any combination between features relating to different subject matters, in particular between features of the apparatus type claims and features of the method type claims is considered as to be disclosed with this application.

### BRIEF DESCRIPTION OF THE DRAWINGS

The aspects defined above and further aspects of the present disclosure are apparent from the examples of embodiment to be described hereinafter and are explained with reference to the examples of embodiment. The disclosure will be described in more detail hereinafter with reference to examples of embodiment but to which the disclosure is not limited.

FIG. 1 shows a schematic view of a device according to an exemplary embodiment of the present disclosure.

FIG. 2 shows a schematic view of a diagram showing detected signals of different wavelengths under pressure variation according to an exemplary embodiment of the present disclosure.

FIG. 3 shows a schematic view of a diagram showing detected signals of different wavelengths under pressure variation and under almost constant pressure according to an exemplary embodiment of the present disclosure.

### DETAILED DESCRIPTION OF EXEMPLARY EMBODIMENTS

The illustrations in the drawings are schematic. It is noted that in different figures similar or identical elements are provided with the same reference signs.

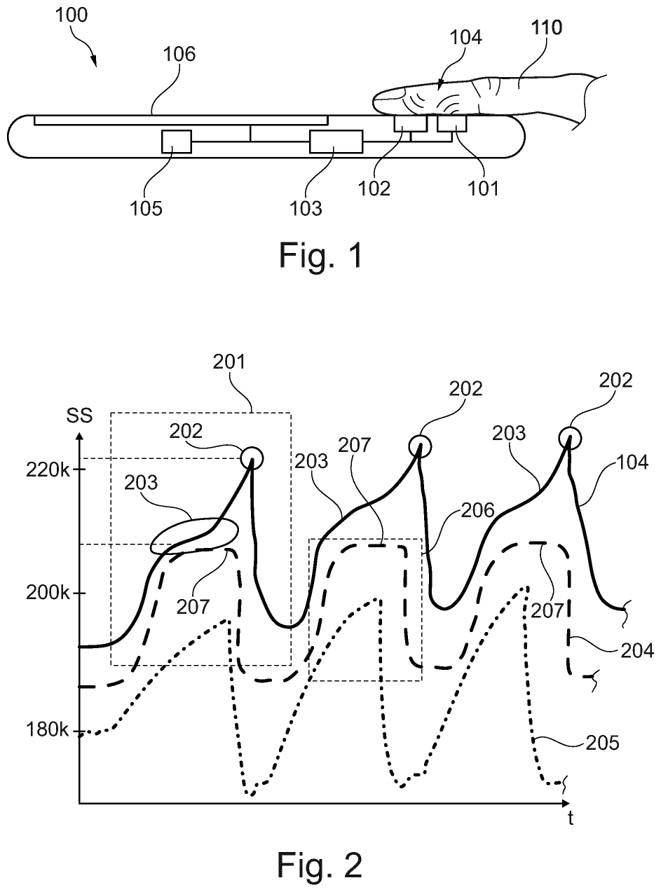

FIG. 1 shows a schematic view of a device according to an exemplary embodiment of the present disclosure. FIG. 2 shows a diagram showing detected signals of different wavelengths under pressure variation by the device according to FIG. 1.

The device 100, such as the shown smartphone, determines a biomarker concentration in a blood of a body part 110, such as the shown fingertip under consideration of the physiological constitution of the body part 110. The device 100 comprises a light source 101 for radiating first light waves 104 to the body part 110, a detector unit 102 for measuring the reflected first light waves 104 reflected from the body part 110 and a processing unit 103 coupled to the detector unit 102 for receiving the measured first light waves 104. The processing unit 103 is configured to determine, at an occurrence of a first specific signal section 202 in a signal profile 201 of the reflected first light waves 104 during a predefined pressure variation applied to the body part 110 by the detector unit 102, at least one characteristic value comprising the signal strength SS of the reflected first light waves 104, wherein the at least one characteristic value at the specific first signal section 202 of the reflected first light waves 104 is representative of a physiological constitution of the body part 110, such that a biomarker concentration in the blood is determinable.

The light source 101 is configured to radiate light with the first wavelength 104 or with a predefined plurality of further wavelengths 204, 205 to the body part. The light source 101 may comprise one or a plurality of LEDs. Specifically, the first wavelength 104 may be infrared light, the second wavelength 204 blue light and the third wavelength 205 green light.

The detector unit 102 may comprise a photodiode which is configured to measure all opposed described spectra used for the respective radiated wavelengths 104, 204, 205. Specifically, the detector unit 102 may detect a picture or the multiple spectra between 410 nm and 1090 nm, for example.

The processing unit 103 may comprise a processor for controlling the light source 101 and the detector unit 102. Specifically, the processing unit may comprise for example an oscillator, a led driver, a temperature sensor and a data register (e.g. a data unit 105). Furthermore, the processing to transfer data via standard buses such as I2C or SPI communications or similar.

Furthermore, the device 100 may comprise a display unit 106 for displaying the measurement results and/or for giving instruction to the user. Additionally, the display unit 106 may form an input unit, such as a touchscreen.

The quality and the quantity of the signal strength of the reflected and hence the detected wavelength 104, 204, 205 is dependent on the physiological constitution of the body part 110 and specifically of the pressure, by which the detection unit 100 is pressed onto the body part 110. However, independent of the applied pressure onto the body part 110 and the physiological constitution of the body part 110, the detected signals during a pre-determined pressure variation can be representative for a quantity of the biomarker concentration.

During the predefined pressure variation in the signal profiles 201, 206 of the detected reflected light waves a specific signal section 202, 207 (e.g. a certain shape) exist during the predefined pressure radiation. Furthermore, it has found out, that the specific signal section 202, 207 and its respective characteristic value (e.g. the strength of the detected signal at the specific signal section 202, 207) is indicative of a certain biomarker (e.g. glucose) and its respective concentration. The value for the signal strength SS may be in the shown example in FIG. 2 between 0 and 224 000 nA. In FIG. 2, the pressure variations and hence the signal strength variations over time t for the wavelengths 104, 204, 205 are shown.

Furthermore, it has found out, that the characteristic values derived from signals of the specific signal sections 202, 207 may define a specific physiological constitution of the body part at the time of measurement. For example, if the body part 110 is a finger and the fingers pressed onto the detection unit 102 during a predefined pressure variation, a local maximum as signal section 202, 207 of a detected signal profile 201, 206 may be indicative of the amount of tissue between the surface of the finger and the bone of the finger. Hence, the thickness of the tissue between the bone and the surface of the finger can be derived which also influence the measurement result of the concentration of the biomarker.

Specific points and specific signal sections 202, 207, respectively, in the signal profile 201, 206 may be a plateau of the signal function, a function break (a sudden change in the slope of the function), a maximum and minimum of the signal function.

The determined characteristic value at specific a specific signal section 202, 207 of the reflected light waves 104, 204, 205 may be compared with existing models comprising the information of a respective biomarker concentration in the blood on the certain characteristic value of a specific signal section 202, 207. The existing models are defined for example in clinical studies and laboratory studies. For example, if the biomarker is glucose, the glucose level and the physiological constitution of the plurality of persons can be measured for example invasively. For example, the exact glucose level may be measured for a specific physiological constitution of the user by an oral glucose tolerance test (OGTT). For measurement value of the glucose level, a specific characteristic value of a specific signal section 202, 207 in the signal profile 201, 206 of the reflected light waves can be determined. Hence, a database, e.g. stored in the data unit 105, comprising a plurality of nominal values can be provided to which the measured characteristic values of the inventive device can be compared with in order to determined specific biomarker concentration the blood. In fact, a plurality of the specific signal sections 202, 203, 207 under a plurality of different light waves can be derived for a specific biomarker concentration under consideration of a specific physiological constitution.

The specific signal section 202 describes for example a maximum. The further specific first signal section 203 describes for example an inflection point. The second signal section 207 of the second signal profile 206 describes for example a plateau of the signal function. Hence, during the predefined pressure variation, the respective signal profiles 201, 206 of the reflected light waves comprise for example the above listed specific signal sections 202, 203, 207 that are indicative for the biomarker concentration and the physiological constitution of the body part 110.

The processing unit 103 is configured to determine on a basis of a plurality of repeated predefined pressure variations occurrences of the first specific signal section 202, 203, 207 in a signal profile 201, 206 of the reflected first light waves for each conducted pressure variation. The processing unit 103 is further configured to determine respective characteristic values of the first specific signal section in each predefined pressure variations and to determine a mean characteristic value of the first specific signal section 202, 203, 207 determined in the predefined pressure variations.

The at least one determined characteristic value, e.g. the signal strength or the slope of the signal, of the specific signal section 202, 203, 207 defines at least one respective characteristical regressors (Rc, Rcf). For example, a signal profile 201, 206 of a reflected wavelength 104, 204, 205 may have a plurality of specific signal sections 202, 203, 207 which may be used as a regressor for defining the regressor relation. Hence, the regressor relation RR is formed by mathematical dependencies and relations of characteristical regressors (Rc) and further characteristical regressors Rcf.

The data unit 105 of the device comprises a data set of predefined regressor relations RR correlated to respective biomarker concentration. The processing unit 103 is further configured to compare the determined regressor relation RR to predefined regressor relations RR, wherein if the determined regressor relation RR is in the vicinity of the predefined regressor relation the biomarker concentration is derivable.

The characteristic values at the appearance of this specific signal sections 202, 203, 207 defines the first list of characteristical regressors Rc, Rcf. This list of regressors Rc, Rcf may be used for generating a regressor relation RR, which can be correlated to a biomarker concentration. The regressor relation RR defines a mathematical relation between at least one characteristical regressor Rc, Rcf or the relation of the plurality of different characteristical regressors. By using statistical methods and machine learning, i.e. artificial intelligence (AI), specific regressor relations can be found out which are suitable for determining by its characteristic value a biomarker concentration for a specific physiological constitution on the basis of the specific signal sections 202, 207 of the signal profiles 201, 206 of reflected light waves under the predefined pressure variation.

Hence, machine learning may be implemented in the evaluation of finding appropriate regressor relation. Detection data of the reflected wavelength 104, 204, 205 captured in laboratory conditions by a detection unit may be at least partially analyzed using machine learning. This may render it possible to obtain highly reliable information concerning regressor relations being indicative of a biomarker concentration under certain physiological constitutions of a specific body part (such as a finger).

The data unit 105 may be implemented in the device 100. However, the data unit 105 may be realized by an input/output interface of the device 100 and the data can be received and/or send to spaced apart data units which store the data.

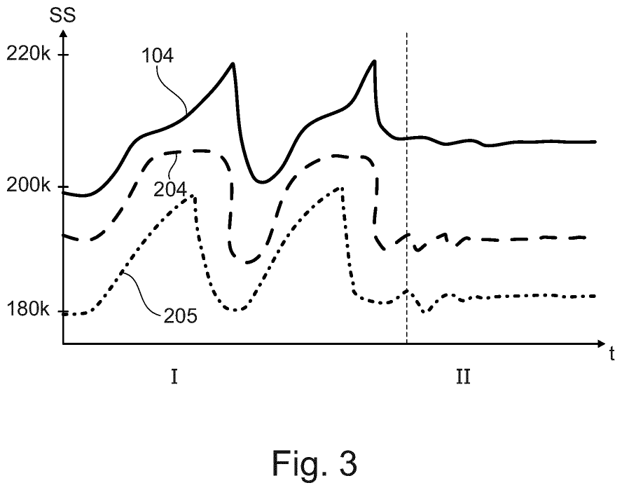

FIG. 3 shows a schematic view of a diagram showing detected signals of different wavelengths 104, 204, 205 under pressure variation I and under almost constant pressure II according to an exemplary embodiment of the present disclosure.

In addition to the above measurement under pressure variation as shown in FIG. 2, the processing unit 103 is further configured to determine at least one measurement value of the signal strength SS of the reflected light waves 104, 204, 205 during a, in particular constant, placement of the detector unit 102 onto the body part 110, wherein the measurement value defines at least one measurement regressor (Rm). The value for the signal strength SS may be in the shown example in FIG. 3 between 0 and 224 000 nA. In FIG. 3, the pressure variations and hence the signal strength variations over time t for the wavelengths 104, 204, 205 under pressure variation measurement I and under non pressure variation measurement II are shown.

The regressor relation (RR) is further determined on the basis of the at least one determined characteristical regressor (Rc, Rcf) and the at least one measurement regressor (Rm). By additionally measuring the reflected wavelengths 104, 204, 205 during a placement of the detector 102 onto the body part, i.e. under (almost) constant pressure, a characteristical value of the reflected signal of a specific wavelength 104, 204, 205 taken under almost constant pressure may define a measurement regressor Rm which can be used for normalization of the data with respect to the present physiological constitution of the body part 110 and with respect to a calibration of the light source 101, e.g. the LEDs. The measurement regressor Rm is additionally considered in the regressor relation RR so that an improved reference of the regressor relation RR to a nominal regressor relation indicative of a bio marker concentration can be achieved.

Summarizing, during a measurement of reflected light of a body part 110 under a predefined pressure variation on the body part, each reflected wavelength 104, 204, 205 (for example red light, infrared light, blue light, green light etc.) has a specific signal profile 201, 206 under a specific pressure variation applied onto the body part. It has found out that each signal profile 201, 206 under pressure variation comprises a respective specific signal section 202, 203, 207 in the signal profile 201, 206 of the reflected first light waves. Furthermore, a specific regressor relation RR can be significantly better correlated to a biomarker concentration (e.g. glucose level) in the blood under consideration of the measurement regressor Rm achieved under an almost constant pressure, shown in section II in FIG. 3.

The specific regressor relation is obtained as mathematical relations of characteristical regressors Rc and e.g. measurement regressors Rm like: Rm1/Rc1, Rm2/Rc1, Rm1/Rc2, Rm1/In(Rc₁), In(Rm₁)/e^{Rc1} etc. The regressor relation RR is correlatable to a biomarker concentration in the blood, such that a determined value of the regressor relation is indicative to a value of the biomarker concentration.

A measurement of a biomarker concentration with the device 100 may be conducted as follows:

When the device 100 is covered with the body part 110 (e.g. the finger pad, preferably an index finger or a ring finger), a fixing element (such as a rubber ring, elastic or any other elastic, rope, fastener) of the device may be optionally used to fix the body part 100 to the device for more accurate measurement.

Next, the measurement of an individual person begins. Each person has a different skin type and other physiological properties which can be evaluated by the device 100. According to the disclosure the skin surface is pushed with e.g. three consecutive pressures to the device, so that blood is squeezed out of the body part 110 (e.g. the tip of the finger) and the body part slightly fades. Pressures occur e.g. in the sequences, first a gradual pressure increase to the point where the (e.g. diode signals are no longer distinguishable, which lasts e.g. about 10 s (seconds), followed by a gradual release of the pressure of e.g. 5 s (see for example signal curves under section I in FIG. 3), and then the whole process can be repeated for example two times or more. Then, the body part may rest e.g. for 20 s onto the device under almost constant pressure (see for example signal curves under section II in FIG. 3).

The instruction for the person may be taken from a display 106 of the device 100 (see FIG. 1).

Next, first the validity of the signal and its quality may be checked. Next, the physiology of the skin of the finger may be considered based on the relationships between above described regressors and the regressor relation RR. The physiology of the body part 110 is considered in the respective regressor relation RR. On the basis of the data of the measurement, it is possible to determine the actual skin and subcutaneous properties on the basis of the first part of the measurement I under pressure variation and perform the physiological normalization (FN) for the second part of the measurement II under almost constant pressure. The physiological normalization is used to normalize the data of the second part of the measurement by translating the values to a neutral (universal) model (databases), where all the values obtained have e.g. the same scale (unit). Based on the data of the regressor relation RR, it is possible to determine the location of the actual measured regressor relation RR within the multidimensional space of the data bases of nominal regressors relations that are correlated to concentration of bio markers, e.g. blood sugar levels.

The location of the measured regressor relation in the multidimensional space of the data bases is determined on the basis of clustering, which, based on the data of the e.g. the first part of the measurement, determines the location of the statistical model in the spectral space of the models. A more detailed classification of the measured regressor relation may be provided by checking the relationships between the signals of different wavelengths in a given measurement range.

It should be noted that the term “comprising” does not exclude other elements or steps and “a” or “an” does not exclude a plurality. Also elements described in association with different embodiments may be combined. It should also be noted that reference signs in the claims should not be construed as limiting the scope of the claims.

### LIST OF REFERENCE SIGNS

100 device
101 light source
102 detector unit
103 processing unit
104 first light waves
105 data unit
106 display
110 body part
201 first signal profile
202 first specific signal section
203 further first specific signal section
204 second light waves
205 third light waves
206 second signal profile
207 second specific signal section
I pressure variation measurement
II non pressure variation measurement
SS signal strength
t time
Rc characteristical regressor
Rcf, Rcf2 further characteristical regressor
Rm Measurement regressor
RR Regressor Relation

1. Device for determining a biomarker concentration in a blood of a body part under consideration of the physiological constitution of the body part, the device comprising
a light source for radiating first light waves to the body part, a detector unit for measuring the reflected first light waves reflected from the body part,
a processing unit coupled to the detector unit for receiving the measured first light waves,
wherein the processing unit is configured to
determine, at an occurrence of a first specific signal section in a first signal profile of the reflected first light waves during a predefined pressure variation applied to the body part by the detector unit, at least one characteristic value comprising the signal strength of the reflected first light waves,
wherein the at least one characteristic value at the specific first signal section of the reflected first light waves is representative of a physiological constitution of the body part, such that a biomarker concentration in the blood is determinable.

2. Device according to claim 1,
wherein the characteristic value further comprises the value of the slope of the signal profile at the occurrence of a specific signal section during the predefined pressure variation applied to the body part by the detector unit.

3. Device according to claim 1,
wherein the first light wave is selected from one of the group comprising infrared light, red light, green light and blue light.

4. Device according to claim 1,
wherein the specific signal section is defined by a characteristic slope, by a plateau of the signal function, a saltus of the signal function, an inflection point, a minimum and a maximum.

5. Device according to claim 1, wherein the processing unit is configured to
determine on a basis of a plurality of repeated predefined pressure variations occurrences of the first specific signal section in a signal profile of the reflected first light waves for each conducted pressure variation,
determine respective characteristic values of the first specific signal section in each predefined pressure variations,
determining a mean characteristic value of the first specific signal section determined in the predefined pressure variations.

6. Device according to claim 1,
wherein the at least one determined characteristic value defines at least one respective characteristical regressor,
wherein the processing unit is configured to
determine a regressor relation on the basis of the at least one determined characteristical regressor,
wherein the regressor relation is correlatable to a biomarker concentration in the blood, such that a determined value of the regressor relation is indicative to a value of the biomarker concentration.

7. Device according to claim 6, further comprising
a data unit comprising a data set of predefined regressor relations correlated to respective biomarker concentration,
wherein the data unit is further configured to
compare the determined regressor relation to predefined regressor relations, wherein if the determined regressor relation is in the vicinity of the predefined regressor relation the biomarker concentration is derivable.

8. Device according to claim 6,
wherein the processing unit is further configured to
determine, at an occurrence of a further first specific signal section in the signal profile of the reflected first light waves during the predefined pressure variation applied to the body part by the detector unit, at least one further characteristic value comprising a further signal strength of the first reflected first light waves,
wherein the at least one further characteristic value at the further specific first signal section of the first reflected first light waves is representative of the physiological constitution of the body part, such that the biomarker concentration in the blood is determinable,
wherein the at least one determined further characteristic value defines at least one respective further characteristical regressor,
wherein the regressor relation is further determined on the basis of the at least one determined further characteristical regressor.

9. Device according to claim 6, wherein the processing unit is further configured to
determine at least one measurement value of the signal strength of the reflected first light waves during a placement of the detector unit onto the body part,
wherein the Measurement Value defines at least one measurement regressor, wherein the regressor relation is further determined on the basis of the at least one determined characteristical regressor and the at least one Measurement regressor.

10. Device according to claim 6,
wherein the light source is configured for radiating second light waves to the body part,
wherein the detector unit is configured for measuring the reflected second light waves reflected from the body part,
wherein the detector unit is configured for receiving the reflected second light waves, and
wherein the processing unit is configured to
determine, at an occurrence of a second specific signal section in a second signal profile of the reflected second light waves during the predefined pressure variation applied to the body part by the detector unit, at least one further characteristic value comprising the signal strength of the reflected second light waves,
wherein the at least one further characteristic value at the specific second signal section of the reflected second light waves is representative of the physiological constitution of the body part,
wherein the at least one determined further characteristic value defines at least one respective further characteristical regressor,
wherein the regressor relation is further determined on the basis of the at least one determined further characteristical regressor.

11. Device according to claim 1, wherein the device is a portable handheld device.

12. Device according to claim 1, wherein the bio marker is Glucose, C-Reactive Protein, Hemoglobin, Cholesterol, LDL, HDL, Fibrinogen and/or Bilirubin.

13. Method of determining a biomarker concentration in a blood of a body part under consideration of the physiological constitution of the body part, the method comprising
radiating first light waves to the body part,
measuring the reflected first light waves reflected from the body part,
determining, at an occurrence of a first specific signal section in a signal profile of the reflected first light waves during a predefined pressure variation applied to the body part by the detector unit, at least one characteristic value comprising the signal strength of the reflected first light waves,
wherein the at least one characteristic value at the specific first signal section of the reflected first light waves is representative of a physiological constitution of the body part, such that a biomarker concentration in the blood is determinable.
